(19) United States
(12) Patent Application Publication
(10) Pub. No.: US 2021/0304858 A1
(43) Pub. Date: Sep. 30, 2021

(54) SECURE CERTIFICATE VALIDATION SYSTEM AND METHOD FOR USE WITH ELECTRONIC HEALTHCARE RECORDS AND OTHER APPLICATIONS

(71) Applicant: Clifton R. LACY, Highland Park, NJ (US)

(72) Inventor: Clifton R. LACY, Highland Park, NJ (US)

(21) Appl. No.: 17/217,762

(22) Filed: Mar. 30, 2021

Related U.S. Application Data
(60) Provisional application No. 63/002,017, filed on Mar. 30, 2020.

(51) Int. Cl. G16H 10/60; G16H 10/40; G06F 21/45; G16H 50/80

(52) U.S. Cl. CPC G16H 10/60; G16H 50/80; G06F 21/45; G16H 10/40

(57) ABSTRACT

Systems and processes for verifying and authenticating a certification or status associated with an individual are described herein. A certificate verification system for COVID-19 verification can include a camera, a database, and a computer system coupled to the camera and the database. The computer system receive a COVID-19 status associated with an individual, wherein the COVID-19 status comprises at least one of a test result or a vaccination status associated with COVID-19, capture, via the camera, an image of the individual, record, in the database, the COVID-19 status and the image associated with the individual, and provide the individual with a record retrieval resource associated with the user profile for retrieving the status and the image of the individual.

### PRIORITY

The present application claims priority to U.S. Provisional Patent Application No. 63/002,017, filed Mar. 30, 2020, titled SECURE CERTIFICATE VALIDATION SYSTEM AND METHOD FOR USE WITH ELECTRONIC HEALTHCARE RECORDS AND OTHER APPLICATIONS, INCLUDING COVID-19 TREATMENT VERIFICATION, which is hereby incorporated by reference herein in its entirety.

### BACKGROUND

Currently, the nation is facing a health crisis in the form of a rapidly spreading virus known as SARS-CoV-2 that causes disease known as COVID-19. Standardized testing and vaccination solutions and encoding have yet to be established nationwide. As a result, those who have already had the virus may be unable to reenter the workforce or rejoin the community, despite being potentially safe. It would be desirable for a technological solution to be provided that leverages a HIPAA-compliant database infrastructure to create a means of tracking the results of those being tested and vaccinated for COVID-19.

Described herein is an application-based solution that securely provides information to an affected individual, and the community, of their COVID-19 test result status. This solution protects private health information while allowing healthcare workers and other essential personnel to safely return to work. This can enable certain business to reopen more quickly and get America back to work. In addition, this solution could be used to identify individuals that have been vaccinated.

### SUMMARY

Described herein may be a certificate verification system (CVS) where accuracy is a matter of life and death. It is desirable for the system to scale to hundreds of millions of people and hundreds of thousands of organizations, comply with HIPAA privacy, and be continuously available. Different parts of the process are managed by different organizations. It is desirable for the system to be deployed very quickly to a large audience. It is desirable that use and operation be fast, simple and self-explanatory. It is desirable for the system to continue to function accurately in the face of extensive attempts to break the system, enter false data, steal data, disrupt operations, and create other problems.

A sample application is certification that an individual has passed one or more tests indicating an immune response to a particular easily communicable disease or to vaccination for the disease. In such a situation, erroneously identifying an immune response that is not present or erroneously failing to identify an immune response that is present can result in serious health and public health consequences.

Many conventional methods of database security, such as passwords, are difficult to use and manage. It is desirable for the system to rely primarily on identifying characteristics available from the individual, such as biometrics, and available resources, such as smart devices. It is desirable for the system to achieve identification that is highly secure. As such, a plurality of techniques can be used in conjunction with complex and novel internal system measures.

One such technique is to exclude extraneous information. Excluding extraneous information simplifies use of the system, eliminates sources of errors, and makes the system more difficult to hack. For example, identifying data, such as an individual's name, address, and phone number require time and training to enter and are sources of misspellings, changes, and other errors that would need to be corrected or changed, thereby adding time and complexity to operation of the system. In addition, such identifying data can be used by hackers to access records in the system. As such, eliminating such data makes finding records, or creating new ones, more difficult.

Another technique is limiting the scope of data for a particular function and eliminating or obscuring information that is not needed for other functions of the verification process. Doing this may eliminate a direct association with data in other parts of the process, thereby making it more difficult to find and use this other data. For example, a collection sample ID is essential for identifying a sample. However, once a test has been performed on the sample, this data can be either deleted or filed in a separate database that is protected separately and is read-only.

Another technique is dealing with many errors by requiring each new sample collected to be tested under a new individual account. Although this can pose a minor annoyance to the individual, such a technique may significantly simplify development and operation of the system by removing a primary path for falsifying data and otherwise interfering with the operation of the system.

One potential use of the system is to provide a readily visible individual Identifier for people who have taken a test. Potential devices include a wristband, identification card, RFID chip, or other device. Such a device can contain identifying information that can be used by the system. Unfortunately, such devices are subject to theft, modification, and counterfeiting. As a result, although such devices may be useful in the operation of the system, it may be desirable for the system to assume that such devices are compromised and compensate for this by implementing separate robust security measures. One potential technique to overcome this concern is to use a plurality of identification mechanisms for an individual. For example, a combination of photographic, facial recognition, fingerprint, voice print, hand geometry, and other biometric data may be used.

In addition to identification mechanisms within the system, a person performing the verification check can apply other techniques to verify a person's identity. For example, the system can display one or more photographs of the individual and rely upon the natural ability of most people to recognize facial characteristics, voices, and other patterns, as part of the identification process.

These techniques may require the system to store various forms of data, such as photographs, voice prints, and fingerprints. Storing such data can create potential paths for hackers to acquire samples from the individual and use them to search for corresponding records in the system. Such data can also provide mechanisms for hackers to modify or delete existing data or insert new data. To protect against this, such data can be protected by a variety of mechanisms, usually at least two for each data item. These mechanisms may include standard methods, such as encryption, that are augmented with atypical and novel techniques, such as including hidden checking information using methods in the broad class of steganography. By using different methods for different data elements, even in the same category, unauthorized data may be readily recognized, and unauthorized searches for matching data may be foiled by disclosing a lack of the correct hidden patterns for that data element. For example, a particular photograph might have two or more check messages hidden within it, where each check message uses a different encoding method. Both the methods and the messages may be selected at random for each data item.

In some embodiments, the present disclosure is directed to a verification system for verifying a status associated with an individual, the system comprising: a camera; a database; and a computer system coupled to the camera and the database, the computer system comprising a processor and a memory, the memory storing instructions that, when executed by the processor, cause the computer system to: receive the status associated with the individual, capture, via the camera, an image of the individual, record, in the database, the status and the image associated with the individual, and provide the individual with a record retrieval resource associated with the user profile for retrieving the status and the image of the individual.

In some embodiments, the present disclosure is directed to a verification system for COVID-19 verification, the system comprising: a camera; a database; and a computer system coupled to the camera and the database, the computer system comprising a processor and a memory, the memory storing instructions that, when executed by the processor, cause the computer system to: receive a COVID-19 status associated with an individual, wherein the COVID-19 status comprises at least one of a test result or a vaccination status associated with COVID-19, capture, via the camera, an image of the individual, record, in the database, the COVID-19 status and the image associated with the individual, and provide the individual with a record retrieval resource associated with the user profile for retrieving the status and the image of the individual.

In some embodiments, the present disclosure is directed to a computer-implemented method for verifying a status associated with an individual, the method comprising: receiving, by a computer system, the status associated with an individual; capturing, via a camera coupled to the computer system, an image of the individual; recording, in a database coupled to the computer system, the status and the image associated with the individual; and providing, by the computer system, the individual with a record retrieval resource associated with the user profile for retrieving the status and the image of the individual.

In some embodiments, the present disclosure is directed to a computer-implemented method for COVID-19 verification, the method comprising: receiving, by a computer system, a COVID-19 status associated with an individual, wherein the COVID-19 status comprises at least one of a test result or a vaccination status associated with COVID-19; capturing, via a camera coupled to the computer system, an image of the individual; recording, in a database coupled to the computer system, the COVID-19 status and the image associated with the individual; and providing, by the computer system, the individual with a record retrieval resource associated with the user profile for retrieving the status and the image of the individual.

In some embodiments, the record retrieval resource comprises at least one of a QR code, a barcode, a biometric identifier, or a unique identifier.

In some embodiments, the record retrieval resource is provided to the individual via at least one of a printed copy, a text message, an email, or a software application executed on a user mobile device.

In some embodiments, the memory stores instructions that, when executed by the processor, cause the computer system to: receive, via a scan of the record retrieval resource by a verification station, an inquiry associated with the individual; and provide the status and the image associated with the individual to the verification station in response to the inquiry.

In some embodiments, the verification station comprises a mobile device, the mobile device comprising a camera to scan the record retrieval resource to initiate the inquiry.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts a diagram of a certificate verification system interacting with a user device and a verification station, in accordance with an embodiment.

FIG. 2 depicts a flow diagram of a process for providing individuals with a record retrieval resource in connection with a certificate verification system, in accordance with an embodiment.

FIG. 3 depicts an illustrative workflow for creating a user profile with a COVID-19 test result status in connection with a certificate verification system, in accordance with an embodiment.

FIG. 4 depicts an illustrative workflow for associating a user profile with a COVID-19 test result status in connection with a certificate verification system, in accordance with an embodiment.

FIG. 5 depicts an illustrative login screen for a software application used in connection with a certificate verification system, in accordance with an embodiment.

FIG. 6 depicts a dashboard screen for a lab facility for the software application used in connection with a certificate verification system, in accordance with an embodiment.

FIG. 7 depicts a user information screen for a lab facility for the software application used in connection with a certificate verification system, in accordance with an embodiment.

FIG. 8 depicts a test result update screen for a lab facility for the software application used in connection with a certificate verification system, in accordance with an embodiment.

FIG. 9 depicts a user status update screen for a lab facility for the software application used in connection with a certificate verification system, in accordance with an embodiment.

FIG. 10 depicts a user status and QR code screen for the software application used in connection with a certificate verification system, wherein the user status is indicated as “pending,” in accordance with an embodiment.

FIG. 11 depicts a user status and QR code screen for the software application used in connection with a certificate verification system, wherein the user status is indicated as “safe,” in accordance with an embodiment.

FIG. 12 depicts a camera scanning a QR code used in connection with a certificate verification system, in accordance with an embodiment.

FIG. 13 depicts the user status retrieved in response to scanning the QR code in FIG. 12 for the software application used in connection with a certificate verification system, in accordance with an embodiment.

### DETAILED DESCRIPTION

As used herein, “COVID-19” means the infectious disease caused by the SARS-CoV-2 virus.

As used herein, “unique identifier” refers to means of identification including QR codes, bar codes, magnetic stripes, radio frequency identification (RFID), and biometrics, among other modalities.

Generally described herein are various systems and processes for verifying that a particular individual has a particular certification or status and authenticating the identity of the individual presenting the certification or status. As used herein, “status” means a state, condition (e.g., a medical or biological condition, such as whether an individual has allergies), or identity associated with an individual. In some embodiments, the systems and processes function by providing individuals having the certification or status with a record retrieval resource that can be used to retrieve their certification or status and biometric information (e.g., an image of the individual) from a centralized database. Accordingly, third parties can scan or read the record retrieval resource to view the individual's certification or status and review the retrieved biometric information to confirm the individual's identity. In some implementations, the systems and processes described herein could be used to confirm an individual's status, defined as the state, condition, or identity of an individual; such as, vaccination status (e.g., vaccinated or not vaccinated) or test results e.g., qualitative [positive or negative] or quantitative) associated with a condition or disease, such as COVID-19.

This system can be used for a wide variety of certifications and other types of information by organizations, such as national governments, states, counties, families, cities, schools, hospitals, churches, community centers, universities, research centers, prisons, military units, companies, country clubs, and individuals.

Types of places which might access the system include restaurants, hotels, hospitals, doctor's offices, museums, apartment buildings, gated communities, movie theaters, theaters, concerts, places with recreational activities (e.g., swimming, tennis, bowling), public transportation, driving services, schools, airports/airlines, office buildings, party spaces, conventions, athletic stadiums, and events of all types.

The system can be extended to multiple types of certifications, including membership in an organization, multiple medical certifications, warnings of allergies, diseases, and other conditions.

The system can be used for temporary certification, such as entry to a particular event, or to perform a delivery or service to a facility with controlled access, such as an apartment building or gated community.

The system could certify that an individual has successfully completed specific training and licensing. The system can maintain a record of quality of service and safety (e.g., ride hailing drivers).

When the system is used for multiple verifications and other information, the certificates and information presented can depend on the identifying information at the verification site. For example, if a facility is required to be free of peanuts or other specific allergens, only the specific results for those conditions would be presented.

For facilities with needs for medical or other conditions, such as hospitals, nursing homes, or intellectual disability facilities, people may be certified to perform specific functions, such as repairs, delivery, or nursing, or to allow entrance to family members and others.

The system can include information regarding vaccinations, immunities to conditions, allergies, sensitivities, and other conditions. Conditions such as the existence of health directives, powers of attorney, and other documents can be included.

The system could provide specific references for an individual. For example, the reference could include information from a particular person that has engaged the individual to perform certain functions and their level of satisfaction with the individual's performance.

Some individuals, such as migrants and refugees, may be concerned about disclosing information, such as their name and address. As such, the system could be limited to include only their medical, security, or other certifications.

In some embodiments, certifications can expire after a given time period. In some embodiments, a certification can be removed or modified based on, for example, updated information. New certifications can be added.

### Certificate Verification System

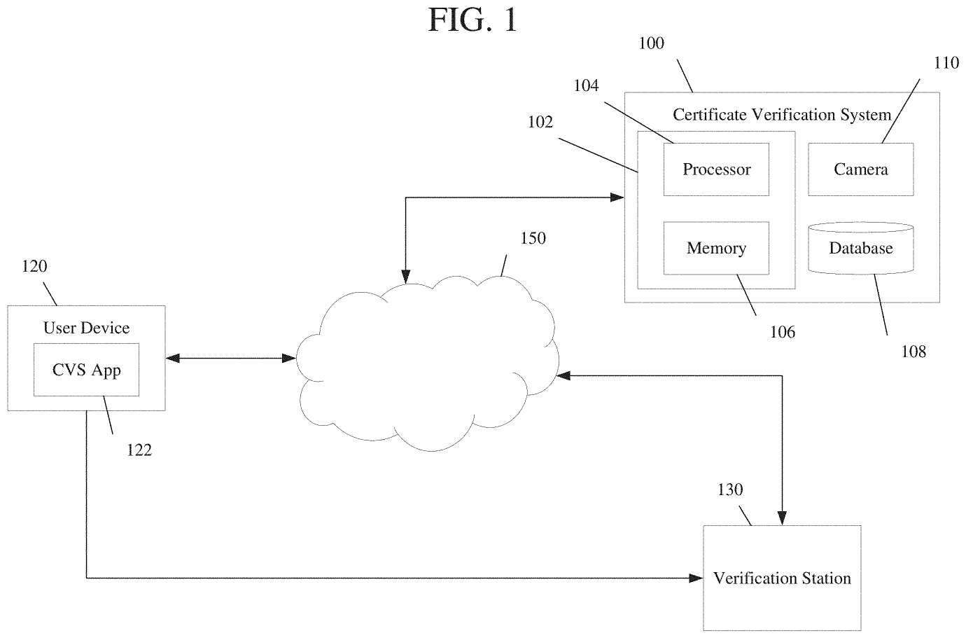

Referring now to FIG. 1, there is shown a diagram of a certificate verification system (CVS) 100 interacting with a user device 120 and a verification station 130, in accordance with an embodiment. In one embodiment, the CVS 100 can include a computer system 102 that is operatively coupled to a biometric capture device and a database 108 for storing user profiles and other records. The biometric capture device can be configured to obtain biometrics or other identifying characteristics associated with an individual that allows the individual to be subsequently identified. In the illustrated embodiment, the biometric capture device includes a camera 110 for capturing an image of the individual. The computer system 102 can include a processor 104 and a memory 106. In one embodiment, the database 108 could be stored locally (i.e., in the memory 106). In another embodiment, the database 108 could be remote from the computer system 102. In some embodiments, the database 108 could be stored in a cloud computing storage system (e.g., Amazon Web Services), a remote server, or any other such remote systems.

In various embodiments, the CVS 100 can be programmed or otherwise configured to receive data pertaining to a status associated with an individual (e.g., a vaccination status or test results), record the status information in association with the individual's biometric information (e.g., an image of the individual) that can be used to subsequently identify the individual, and provide the individual with a record retrieval resource that third parties can scan or use to retrieve the individual's status and biometric information for verifying the individual's status. In various embodiments, the CVS 100 can be associated with or accessible by lab or testing facilities, healthcare providers, hospitals, and other providers of vaccines or tests so that the providers can update the individual's status with respect to the vaccines and/or tests. For example, a healthcare provider could administer a vaccine (e.g., a COVID-19 vaccine) to an individual, take a picture of the individual via a camera 110, and upload the individual's vaccination status (i.e., the fact that the individual has been vaccinated) and the image of the individual to the computer system 102 for storage in and subsequent retrieval from the database 108. Further, the CVS 100 can provide the individual with a record retrieval resource that can be subsequently scanned by a third party to retrieve the individual's vaccination status and image so that the third party can verify the individual's vaccination status and identity. In various embodiments, the record retrieval resource could include a QR code (or other barcode), an RFID tag, or any other scannable identifiers that can embody information. In one embodiment, the record retrieval resource could be provided to the individual via email (e.g., within the body of the email or via a uniquely generated secure URL contained within the email), a text message (e.g., via a uniquely generated secure URL), or a software application 122 executed on a user device (e.g., a mobile device, a tablet, a laptop, or a desktop computer) that is associated with or communicatively connectable to the CVS 100. In other embodiments, the record retrieval resource could be provided on a wristband or another wearable article, an ID card, or any other physical object that could be presented by the individual for scanning. The record retrieval resource can be scanned by a third party at a verification station 130 to retrieve the individual's status information and biometric information. In various embodiments, the verification station 130 can include a dedicated terminal configured to read or scan the record retrieval resource, a mobile device or another device including a camera (e.g., to scan the QR code to retrieve the individual's information), and so on.

Accordingly, the CVS 100 provides a centralized, reliable way to store status information (e.g., vaccination status) in association with biometric information that allows the individual to be identified to confirm that the individual presenting the record retrieval resource is in fact the correct individual associated with the status information. This allows third parties to identify individuals and confirm their status information in a much more reliable and secure manner than conventional techniques (e.g., vaccination cards).

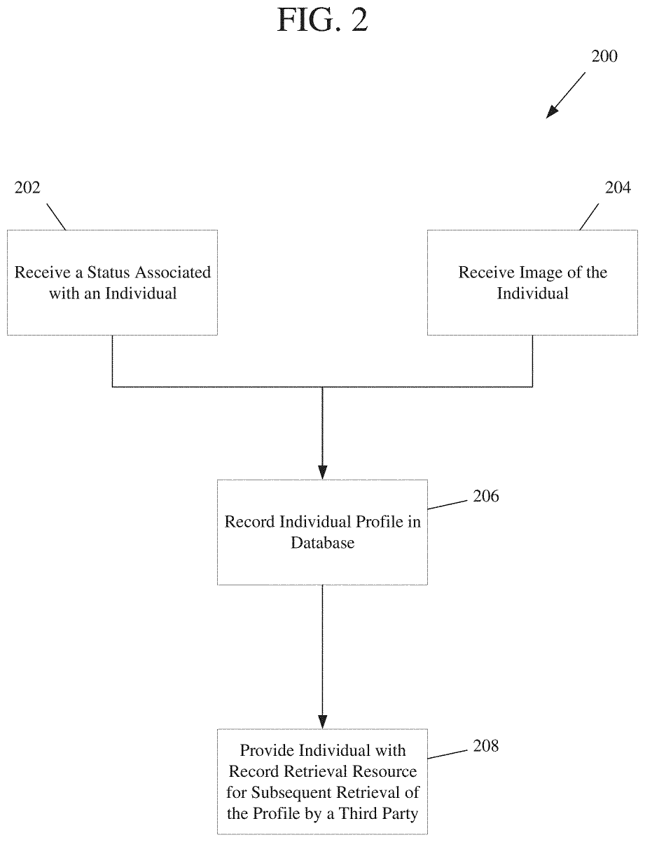

In one embodiment, systems, such as the verification system 100 described above, can be configured to execute various processes for providing individuals with record retrieval resources in order to allow them to confirm their certification or status (e.g., vaccination status) with third parties. One example of such a process 200 is shown in FIG. 2. In the following discussion of the process 200, reference should also be made to FIG. 1. In one embodiment, the process 200 can be embodied as instructions stored in a memory 106 that, when executed by a processor 104, cause the computer system 102 to perform the process. In various embodiments, the process 200 can be embodied as software, hardware, firmware, and various combinations thereof.

Accordingly, the computer system 102 executing the process 200 receives 202 a status associated with an individual. In various embodiments, the status could include a vaccination status or test results associated with the individual. In one embodiment, the status could be input by a third party that is providing testing or other care services to the individual. For example, the status could be input by a laboratory technician at a testing facility, a healthcare provider administering vaccines, and so on. The status could be input by the party via a terminal (e.g., a computer, mobile device, or tablet) via a software application (e.g., the CVS app 122 described above in connection with FIG. 1) that is communicatively coupled to the CVS 100. In one embodiment, the computer system 102 further receives 204 an image of the individual. In other embodiments, the computer system 102 could receive other biometric information, in addition to or in lieu of the image, that could be used to subsequently identify the individual, such as a fingerprint.

Accordingly, the computer system 102 records 206 a profile for the individual in the database 108. The user profile can include the status information and the biometric information (e.g., the image of the individual). In one embodiment, the user profile can be automatically created by the computer system 102 upon input of the data. In another embodiment, the user profile can be created by the user or another party and the status information and biometric information can be associated with the user profile via, for example, a user ID or PIN associated with the profile. In some embodiments, the user profile can include additional information, such as bibliographic information associated with the individual.

Accordingly, the computer system 102 provides 208 the individual with a record retrieval resource for subsequent retrieval of the user profile. In one embodiment, the record retrieval resource could include a QR code that, when scanned, causes a verification station 130 to retrieve the corresponding user profile from the CVS 100. In various embodiments, the record retrieval resource could include a QR code (or other bar code), an RFID tag, and so on. As noted above, the record retrieval resource could also be provided to the individual in a variety of different manners, such as via email, a text message, or a physical object (e.g., an ID card or a wristband).

After creation of the user profile, the computer system 102 can be further configured to receive queries associated with the user profile (e.g., via scanning of the record retrieval resource) and provide the information stored in association with the user profile in response thereto. In some embodiments, the computer system 102 could also receive biometric information captured in association with the individual (e.g., at the verification station 130). The computer system 102 could be further configured to match the received biometric information with the information stored in the user profile as an initial check to confirm the individual's identity.

It should be noted that although the steps of the process 200 are depicted and described in a particular order, this is simply for illustrative purposes. Various steps of the process 200 could be performed simultaneously with each other or in a different order, as will be appreciated by a person skilled in the technical field. The present disclosure is intended to cover all such modifications and variations of the process 200.

### Use Cases

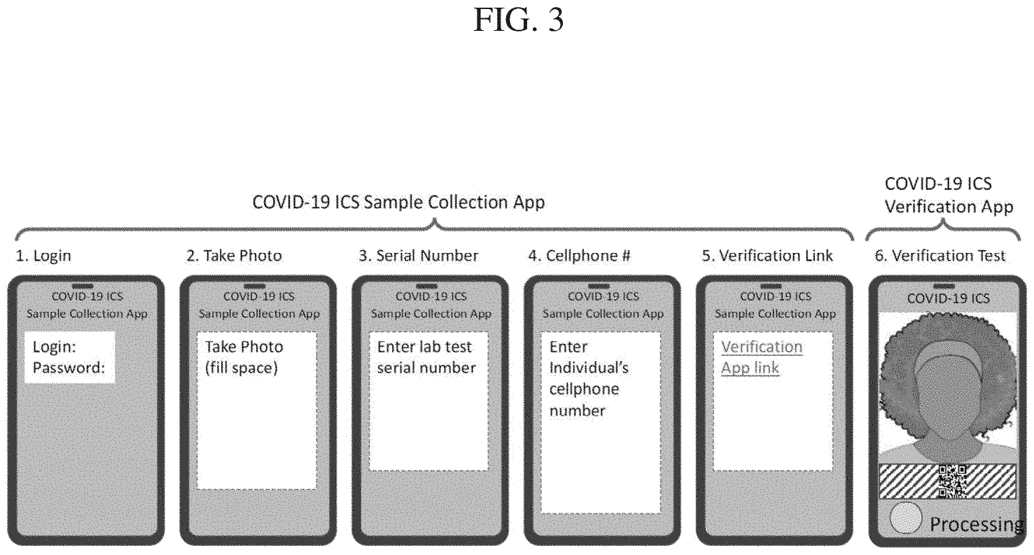

FIG. 3 depicts an illustrative workflow for creating a user profile with a COVID-19 test result status in connection with a CVS 100, in accordance with an embodiment. In the depicted embodiment, the CVS 100 is used as an individual certification system (ICS) for COVID-19. In some embodiments, different software applications can be used for different purposes and interact with the CVS 100 in different manners. For example, lab testing personnel could use an app in conjunction with sample collection to input the individual's biometric information (e.g., take a photo of the individual), enter the lab testing information, enter the individual's contact information (e.g., email address or telephone number), and send the individual a verification link to verify their information (e.g., via an email or text message confirmation link). Further, a verification app could be used to access the record retrieval resource associated with the individual's user profile and the biometric information.

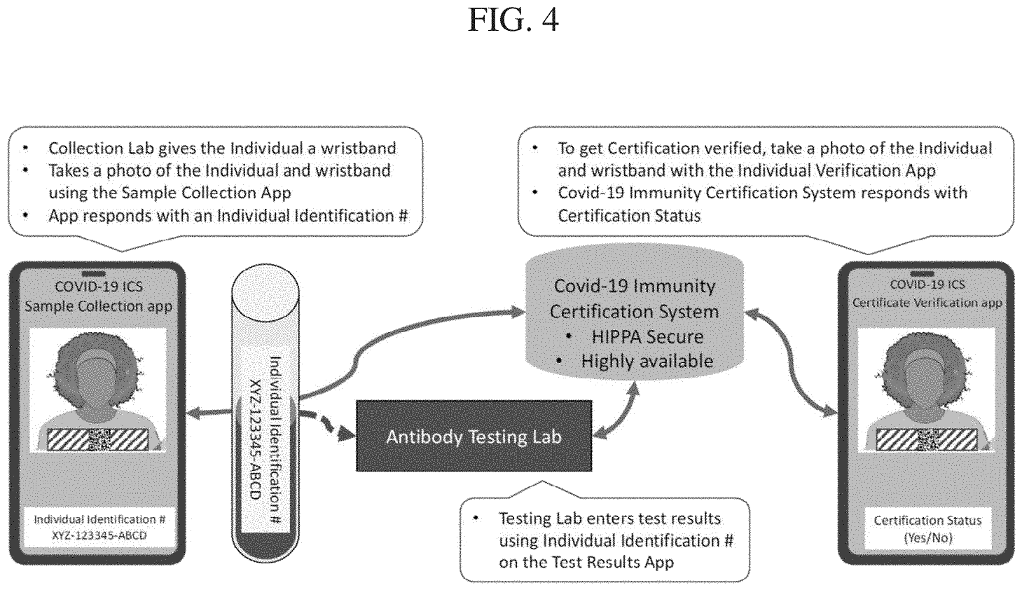

FIG. 4 depicts an illustrative workflow for associating a user profile with a COVID-19 test result status in connection with a certificate verification system, in accordance with an embodiment. In this embodiment, a lab technician can take a sample from the individual, perform a test (e.g., an antibody test for COVID-19), and input the test results or other information via the aforementioned sample collection app. Further, the individual can use the verification app to access their biometric information, certification or status based on the input test results, and/or record retrieval resource for presenting to third parties to confirm their certification or status.

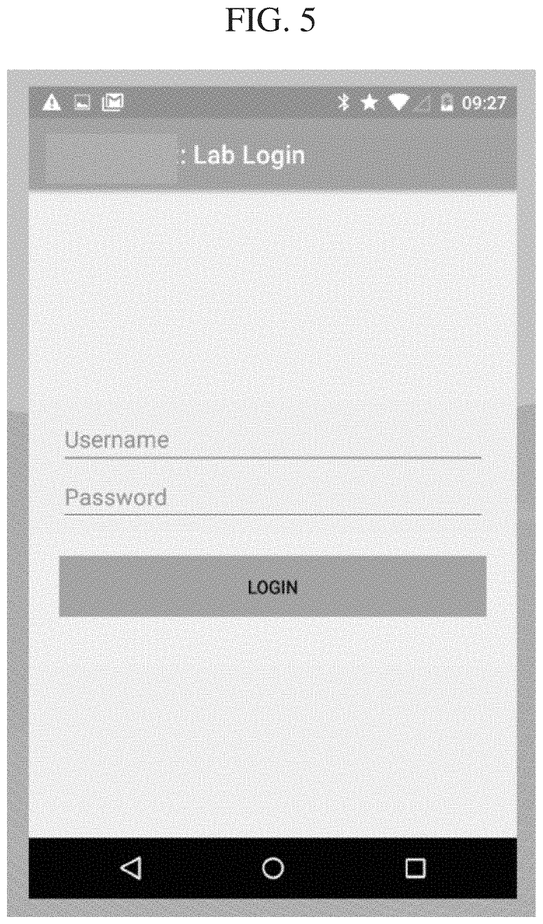

FIGS. 5-13 show embodiments of various screens in the apps described above. For example, FIG. 5 depicts an illustrative login screen for a sample collection app for use with the CVS 100.

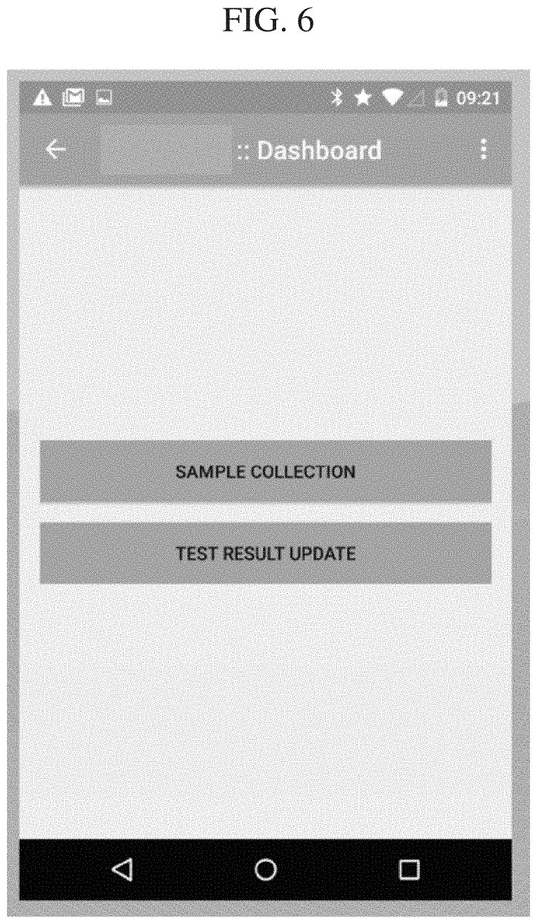

FIG. 6 depicts an illustrative dashboard screen for the sample collection app for use by a lab facility. As can be seen, the dashboard screen can either be used to initiate the sample collection process or update a previously initiated test result.

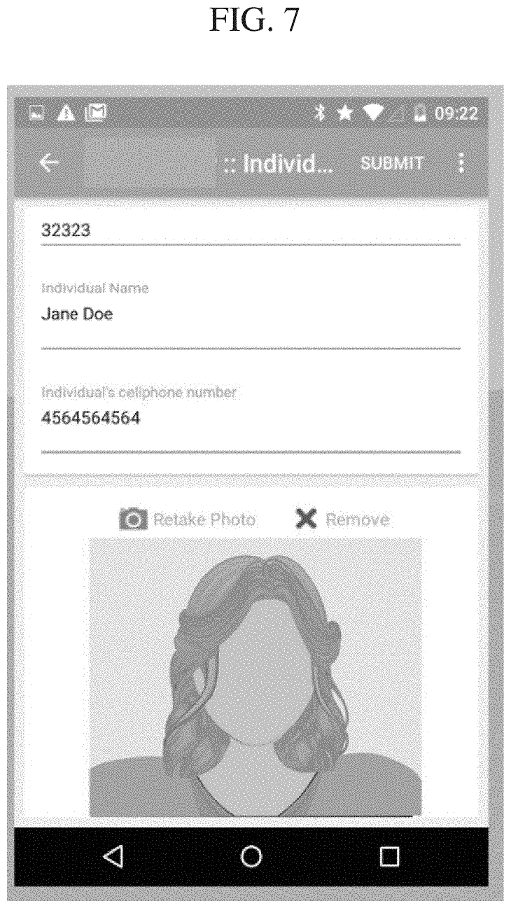

FIG. 7 depicts an illustrative user information screen for the sample collection app. As can be seen, the user information screen could be used to input information associated with the individual and the biometric information that is subsequently used to identify the individual.

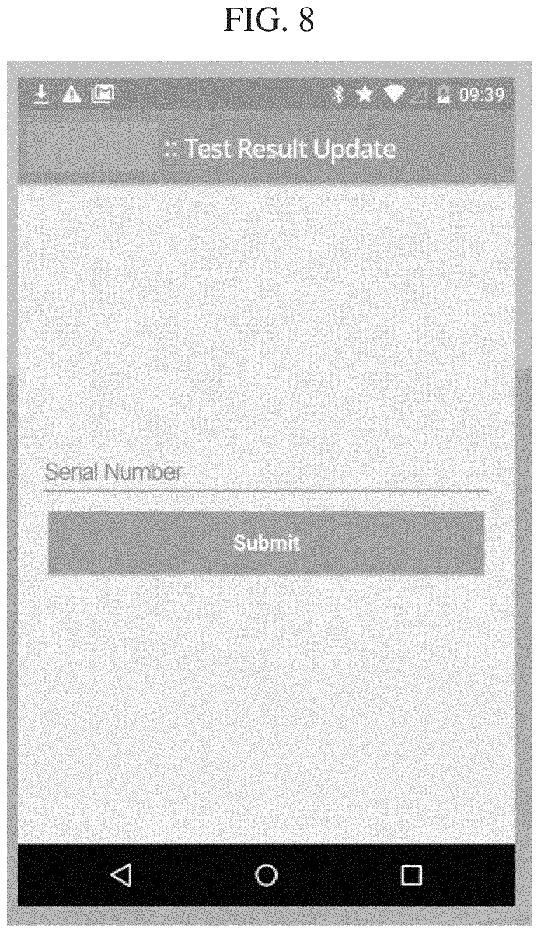

FIG. 8 depicts an illustrative test result update screen for the sample collection app, which allows a lab technician to input identifying information, such as the test serial number, for the test being performed for the individual.

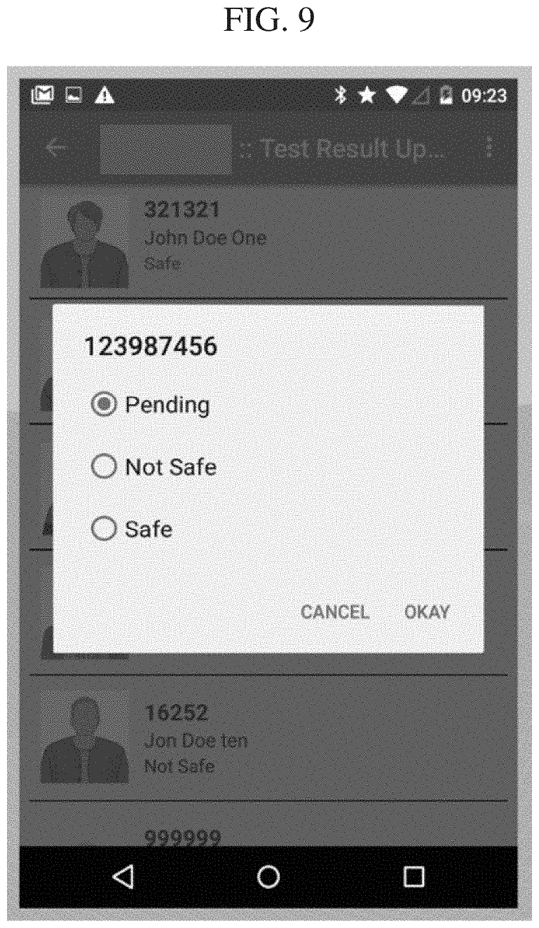

FIG. 9 depicts an illustrative user status update screen for the sample collection app. As shown, the user status update screen allows the lab technician to enter one or more statuses or certifications associated with the test results for the individual. In this particular example, the lab technician has entered that the individual's test results are still pending.

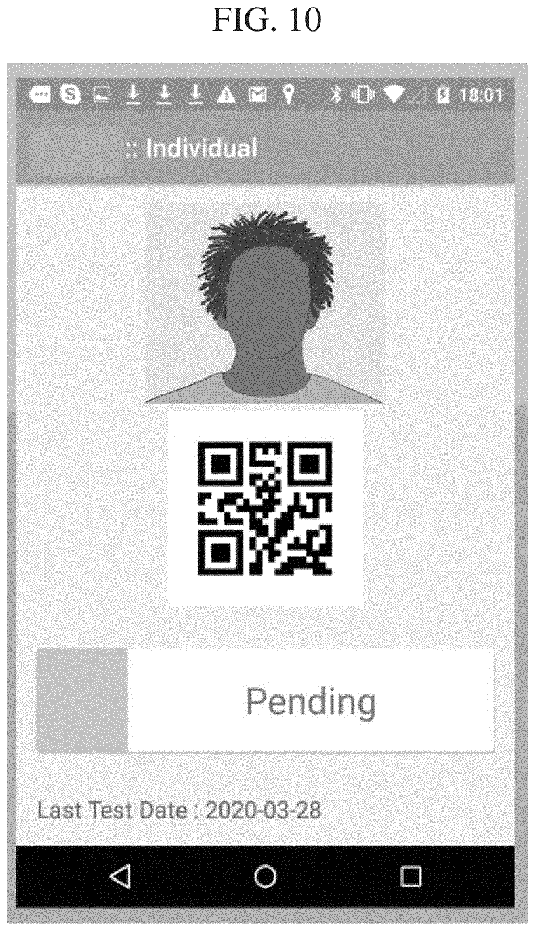

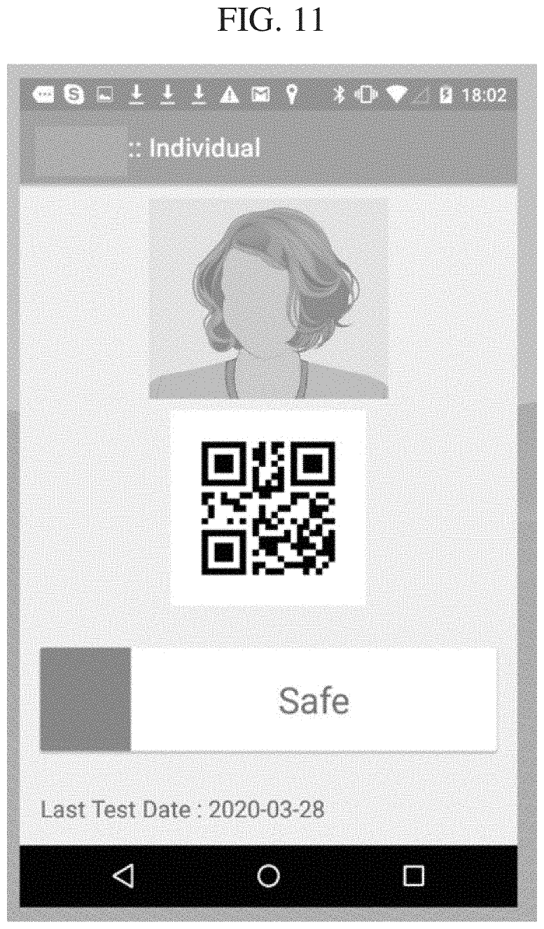

FIGS. 10 and 11 depict illustrative user profile screens for the verification app, which show the user's biometric information, status or certification, and the record retrieval resource that can be scanned by third parties (e.g., at a verification station 130) to retrieve and verify the individual's status.

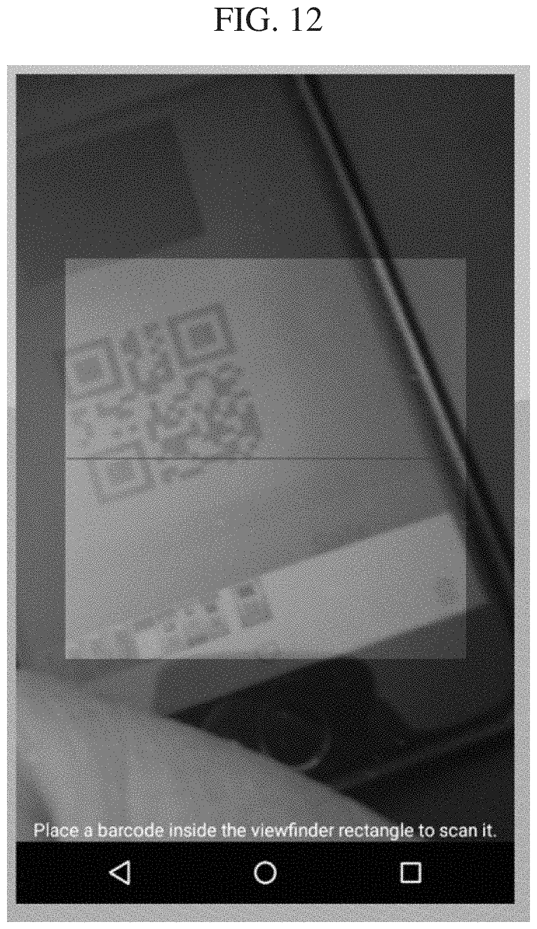

FIG. 12 depicts a camera scanning a QR code used in connection with the CVS 100. As described above, the record retrieval resource can be scanned (e.g., at a verification station 130) and, once scanned, retrieves the user profile associated therewith.

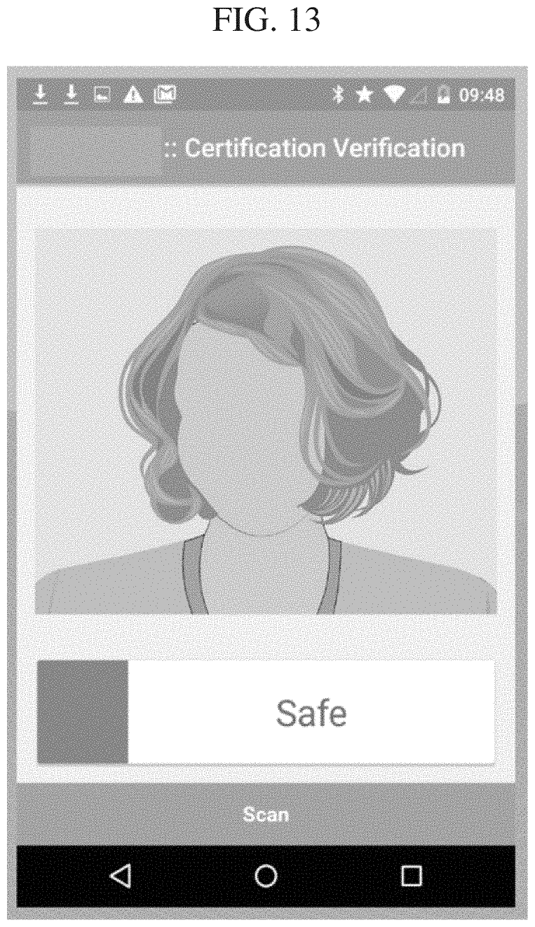

FIG. 13 depicts an illustrative user status screen retrieved in response to scanning the QR code in FIG. 12. Accordingly, the third party scanning the record retrieval resource can confirm the individual's status and, because the biometric information is presented in conjunction with the status information, also confirm that the individual presenting the record retrieval resource is in fact the correct individual for the given user profile.

As described above, one illustrative application of the systems and processes described above includes a wristband that can be used as a visible identification of the individual's status or certification. In one embodiment, the wristband could have both a visible QR code and also two or more hidden, randomly selected patterns in other parts of the wristband (such locations may also be randomly selected, including in the QR code). A photograph of the individual is taken as part of the collection process. This photograph has two or more randomly selected messages hidden within it using two or more randomly selected methods of forming the messages. For example, steganography has many different methods for hiding data within a photograph that are not visible, such as slightly changing the color of selected pixels, especially using colors that are not particularly visible to the human eye, but are detectable by a smart device camera. Alternatively, altering the least significant bit in selected pixel colors may be readily detected in the digital representation of the photograph, but essentially invisible when displayed, especially on a smart device screen. Similar techniques can be applied to voice prints, such as subtle frequency shifts, alterations in the least significant bits, etc.

One illustrative procedure to verify that an individual has a particular status (e.g., vaccinated) or has passed a particular test is as follows: (1) The individual approaches the verification site (e.g., which has a verification station 130); (2) the verification site smart device (e.g., the verification station 130) is running the aforementioned verification app and displays an identifying code; (3) the verification app on the individual's smart device recognizes the identifying code and sends a message to the CVS 100; (4) the CVS 100 sends a message to the verification site smart device with information about the individual; (5) the verification site smart device instructs the individual to present the wristband having the record retrieval resource for decoding; (6) the verification site smart device sends the wristband information to the system; (7) the verification site smart device instructs the individual to pose for a photograph; (8) the verification site smart device sends the photograph to the system; (9) the verification site smart device instructs the individual to say a phrase; (10) the verification site smart device sends the recorded phrase to the CVS 100; (11) the CVS 100 compares the wristband data, the photograph, and the voice recording with the stored information to determine a match; (12) if they match, the CVS 100 sends the stored photograph and the individual's test status to the verification site smart device; (13) if the test result is positive, the person at the verification site compares the photograph on the verification site smart device with the individual's appearance; (14) if they match, the person is authorized for whatever the verification site offers, for example, entry to a restaurant; and (15) if any of these checks fail, access to the verification site is denied and a record of the problem is made in the system for subsequent action, such as invalidating the individual's record, changing the test status to denote an error, or the like.

One illustrative procedure to create a certification for an individual is as follows: (1) the lab collects a blood sample or other bodily fluid sample from the individual; (2) the lab determines COVID-19 immunity for the individual from the blood sample or other bodily fluid sample; (3) if the individual is immune, a profile is created for the individual in a COVID-19 database (e.g., the database 108); (4) issue an identification device (e.g., a wristband) to the individual; and (5) subsequently the identification device can be scanned or read to check the test certification verification for the individual.

One illustrative procedure to initialize a user's mobile device with the software app is as follows: (1) download the COVID-19 certification verification app to the smart device; (2) click “Register Device” on the smart device; (3) send information from the smart device to the CVS 100; and (4) have the CVS 100 test the device by registering the device or rejecting the device and providing instructions to the user.

One illustrative procedure to verify an individual's certification status is as follows: (1) the individual approaches a verification station 130; (2) the individual presents the record retrieval resource and/or identification; (3) the verification station 130 sends the information to the CVS 100; (4) the CVS 100 checks the certification of the individual; (5) the CVS 100 sends a response to registration station 130 (e.g., “Certified” or “Not Certified”); (6) if certified, the individual is allowed to proceed; (7) if not certified, the individual is turned away (and, in some cases, additional “Not Certified” processes can be implemented); and (8) the results of all entries, changes, queries, responses, and accesses are stored in the database 108.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

The functions and process steps herein may be performed automatically or wholly or partially in response to user command. An activity (including a step) performed automatically is performed in response to one or more executable instructions or device operation without user direct initiation of the activity.

What is claimed is:

1. A verification system for COVID-19 verification, the system comprising:
a camera;
a database; and
a computer system coupled to the camera and the database, the computer system comprising a processor and a memory, the memory storing instructions that, when executed by the processor, cause the computer system to:
receive a COVID-19 status associated with an individual, wherein the COVID-19 status comprises at least one of a test result or a vaccination status associated with COVID-19,
capture, via the camera, an image of the individual,
record, in the database, the COVID-19 status and the image associated with the individual, and
provide the individual with a record retrieval resource associated with the user profile for retrieving the status and the image of the individual.

2. The system of claim 1, wherein the record retrieval resource comprises at least one of a QR code, a barcode, a biometric identifier, or a unique identifier.

3. The system of claim 1, wherein the record retrieval resource is provided to the individual via at least one of a printed copy, a text message, an email, or a software application executed on a user mobile device.

4. The system of claim 1, wherein the memory stores instructions that, when executed by the processor, cause the computer system to:
receive, via a scan of the record retrieval resource by a verification station, an inquiry associated with the individual; and
provide the status and the image associated with the individual to the verification station in response to the inquiry.

5. The system of claim 4, wherein the verification station comprises a mobile device, the mobile device comprising a camera to scan the record retrieval resource to initiate the inquiry.

6. A computer-implemented method for COVID-19 verification, the method comprising:
receiving, by a computer system, a COVID-19 status associated with an individual, wherein the COVID-19 status comprises at least one of a test result or a vaccination status associated with COVID-19;
capturing, via a camera coupled to the computer system, an image of the individual;
recording, in a database coupled to the computer system, the COVID-19 status and the image associated with the individual; and
providing, by the computer system, the individual with a record retrieval resource associated with the user profile for retrieving the status and the image of the individual.

7. The method of claim 6, wherein the record retrieval resource comprises at least one of a QR code, a barcode, a biometric identifier, or a unique identifier.

8. The method of claim 6, wherein the record retrieval resource is provided to the individual via at least one of a printed copy, a text message, an email, or a software application executed on a user mobile device.

9. The method of claim 6, further comprising:
receiving, by the computer system from a scan of the record retrieval resource by a verification station, an inquiry associated with the individual; and
providing, by the computer system, the status and the image associated with the individual to the verification station in response to the inquiry.

10. The method of claim 9, wherein the verification station comprises a mobile device, the mobile device comprising a camera to scan the record retrieval resource to initiate the inquiry.
